SEQUENCE LISTING (22 sequences)

SEQ ID NO: 1 - DNA, 20 bp, Artificial Sequence
  Oligonucleotide primer - ATP-A
aaacaagctgcccacatagg

SEQ ID NO: 2 - DNA, 20 bp, Artificial Sequence
  Oligonucleotide primer - ATP-A
cagcaagacaagctgacaga

SEQ ID NO: 3 - DNA, 21 bp, Artificial Sequence
  Oligonucleotide primer - ATP-B
cctgccgctcttctttatcgg

SEQ ID NO: 4 - DNA, 19 bp, Artificial Sequence
  Oligonucleotide primer - ATP-B
accagatggctgcacgttg

SEQ ID NO: 5 - DNA, 19 bp, Artificial Sequence
  Oligonucleotide primer - ATP-C
cacttggaggcacctcaca

SEQ ID NO: 6 - DNA, 20 bp, Artificial Sequence
  Oligonucleotide primer - ATP-C
acaagcccaagtagaccctt

SEQ ID NO: 7 - DNA, 19 bp, Artificial Sequence
  Oligonucleotide primer - ATP-D
caggccatgctctccgaaa

SEQ ID NO: 8 - DNA, 19 bp, Artificial Sequence
  Oligonucleotide primer - ATP-D
agccaccactgcaatctcc

SEQ ID NO: 9 - DNA, 21 bp, Artificial Sequence
  Oligonucleotide primer - ATP-E
ccctgggaatccagtggattg

SEQ ID NO: 10 - DNA, 22 bp, Artificial Sequence
  Oligonucleotide primer - ATP-E
agcaggctatggcaaagaagag

SEQ ID NO: 11 - DNA, 22 bp, Artificial Sequence
  Oligonucleotide primer - ATP-F
ggaaattcctcaggcatagtgg

SEQ ID NO: 12 - DNA, 21 bp, Artificial Sequence
  Oligonucleotide primer - ATP-F
ctgggattcctgcaagaactc

SEQ ID NO: 13 - DNA, 23 bp, Artificial Sequence
  Oligonucleotide primer - ATP-8
gggaatcataaccattcctacgg

SEQ ID NO: 14 - DNA, 22 bp, Artificial Sequence
  Oligonucleotide primer - ATP-8
gaggatttgcatcctgctagac

SEQ ID NO: 15 - DNA, 20 bp, Artificial Sequence
  Oligonucleotide primer - AT1
ctgatggcttctcacaacac

SEQ ID NO: 16 - DNA, 20 bp, Artificial Sequence
  Oligonucleotide primer - AT1
ccgactcaatgaagaaccag

SEQ ID NO: 17 - DNA, 20 bp, Artificial Sequence
  Oligonucleotide primer - AT2
gctggttttaccatcatcgt

SEQ ID NO: 18 - DNA, 20 bp, Artificial Sequence
  Oligonucleotide primer - AT2
gactcaggccgtaataggag

SEQ ID NO: 19 - DNA, 20 bp, Artificial Sequence
  Oligonucleotide primer - AT3
aagtgacctgttccggatac

SEQ ID NO: 20 - DNA, 20 bp, Artificial Sequence
  Oligonucleotide primer - AT3
ccatacctgtttgcctgatg

SEQ ID NO: 21 - DNA, 20 bp, Artificial Sequence
  Oligonucleotide primer - AT4
ggcgttatctccattgcttc

SEQ ID NO: 22 - DNA, 20 bp, Artificial Sequence
  Oligonucleotide primer - AT4
gagattggaacccagtctct

(19) United States
(12) Patent Application Publication
(10) Pub. No.: US 2007/0148703 A1
(43) Pub. Date: Jun. 28, 2007

(54) Method of screening remedy for breast cancer

(75) Inventors: Ikumi Tamai, Chiba (JP); Takashi Nozawa, Chiba (JP)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Genomembrane, Inc., Yokohama-shi, Kanagawa (JP)

(21) Appl. No.: 10/561,504

(22) PCT Filed: Jun. 18, 2004
(86) PCT No.: PCT/JP04/08958

§ 371 Date: Dec. 11, 2006

(30) Foreign Application Priority Data
Jun. 20, 2003 (JP) 2003-177021

(51) Int. Cl. G01N 33/567; A61K 31/56

(52) U.S. Cl. 435/7.2; 514/182

(57) ABSTRACT

The present invention is a method for screening a remedy for breast cancer which does not require uptake into cells unlike aromatase inhibitors, estrogen sulfatase inhibitors, or estrogen competitive inhibitors such as tamoxifen and toremifene, and which shows excellent drug delivery property and extremely rare adverse effect. In the method, a remedy for breast cancer which inhibits transporter function is screened by the process comprising the steps of: culturing a cultured human breast cancer cell line such as MCF-7 cell line or T-47D cell line in the presence of estrone-3-sulfate and a test substance such as bromosulfophtalein; and evaluating whether the uptake of estrone-3-sulfate into cells via estrone-3-sulfate transporters expressed on the cell surface is inhibited, and whether the proliferation of MCF-7 cell line is suppressed.

### TECHNICAL FIELD

The present invention relates to a method for screening a remedy for breast cancer wherein the level of inhibition of transporter activity of estrone-3-sulfate transporter is measured/evaluated.

### BACKGROUND ART

Membrane proteins, etc., which transport materials intra- and extracellularly, are called transporters. It is known that when a specific molecule binds to a transporter embedded in a lipid bilayer membrane, the conformation of the transporter changes and uptake or efflux transport of materials occur. In recent years, genes of such transporters, for example, organic anion transporters such as OAT1 which transport organic anion materials, organic cation transporters such as OCT 1 which transport organic cation materials, peptide transporters such as PEPT 1 which transport peptide materials, have been successively isolated/identified. Some of these transporter genes are eccentrically located in normal tissues/organs throughout the body, however, some of them are known to be locally located in specific tissues/organs such as kidney, liver, brain, etc.

On the other hand, estrogen is what is called a female hormone which causes estrous phenomena in female animals, and is classified into naturally-occurring estrone (E1), estradiol (E2), estriol (E3), estetrol (E4), and synthetic estrogen having bioactivity similar to that of the natural ones. Except for some synthetic ones such as stilbestrol, estrogen has a steroid structure. Biological effect of 0.1 μg of estrone is set at 1 IU (international unit), and this is used as a unit of estrogen. Estrogen is secreted mainly from the follicle and the corpus luteum in the ovary, however, it is secreted also from the fetoplacental unit in pregnancy, the paranephros, the testis, etc. Though the estrogen secretion from the ovary is controlled by gonadotropic hormone secreted from the adenohypophysis (descending regulation), the feedback effect of estrogen on diencephalohypophysial system is also observed (ascending regulation), and therefore, it is said that the sexual cycles are established by the correlation between the two regulations.

In addition, estrogen exerts its action through its receptor, and its action affects not only target tissues, which are diencephalon-adenohypophysis-genital organ and mammary gland, but the entire body. Main physiological effects of estrogen include proliferation of endometrium, growth of uterine muscle, appearance of secondary sexual characters, mediation of the establishment of menstrual cycle, induction of maternal changes in pregnancy, and promotion of proliferation and secretion of mammary gland duct. Estrogen is metabolized mainly in the liver, becomes conjugated estrogen such as estrone-3-sulfate, and then is excreted into the urine. Estrogen is clinically used for the treatment of amenorrhea, menstrual abnormalities, artificial control of the menstruation, menopausal discomfort, hormonal therapy for prostate cancer or breast cancer, and for osteoporosis, etc.

Breast cancer is a malignant tumor which originates in a peripheral mammary duct in an epithelial tissue of mammary gland and acinar epithelium, and is the first leading cause of cancer death for females in Europe and the United States. Though occurrence frequency is much lower in Japan, breast cancer mortality is increasing with the westernized diet and lifestyle changes in recent years. Breast cancer occurs in women, more often in their forties. Examples of the risk factor include: personal/family history of breast cancer, unmarried, late primiparity, early menophania and late menopause, obesity, exposure to radiation, high-fat diet, and history of benign breast diseases. Lymph node metastasis occurs frequently in axilla, subclavian, parasternum, and the number of metastasis correlates with prognosis. Hematogenous metastasis occurs often within the bone, the lung, the liver. Main symptom is breast mass which shows irregular surface, hardness, indefinable boundary and less mobility.

Two-thirds of breast cancers are estrogen-dependent (N Engl J Med 302: 78-90, 1980), i.e., their cell proliferation is regulated by estrogens. Breast cancer exhibits abnormal actions of estrogen receptor system from the beginning of carcinogenesis, and though it continues estrogen-dependent proliferation in its early stage, the proliferation gradually gets out of such control. Judging from the above, it is considered that estrogen and its receptor are deeply involved in the onset and progression of breast cancer, however, the mechanism has not been elucidated. The biologically active form of estrogen is estradiol, which is synthesized through two main pathways (J Clin Endocrinol Metab 57: 1125-1128, 1983; Breast Cancer Res Treat 7: 35-44, 1986). One is the aromatase pathway, in which aromatase converts androgens to estrogens, and the other is the sulfatase pathway, in which steroid sulfatase converts estrone-3-sulfate to estrone. Since sulfatase activity is 50-200 times higher than aromatase activity in pre- and post-menopausal patients with breast cancer, the sulfatase pathway is the major source of biologically active estrogen derived from estrone-3-sulfate (Ann N Y Acad Sci 464: 126-137, 1986; J Clin Endocrinol Metab 81: 1460-1464, 1996). Moreover, the circulating plasma concentration of estrone-3-sulfate is about 10-2,000 times higher than that of unconjugated estrogens and the half-life of estrone-3-sulfate is longer than that of estradiol (J Clin Endocrinol Metab 81: 1460-1464, 1996; J Biol Chem 236: 1043-1050, 1961; J Clin Invest 51: 1020-1033, 1972). Therefore, estrone-3-sulfate is thought to play an important role in the progression of breast cancer as a reservoir of active estrogen, even though estrone-3-sulfate itself is not biologically very active.

The stimulation of proliferation of breast cancer cells by estrone-3-sulfate involves the following sequence of processes: uptake of estrone-3-sulfate into the cells, desulfation by estrogen sulfatase, conversion of estrone to estradiol by 17β-hydroxysteroid dehydrogenase type I, binding to nuclear estrogen receptor, and the regulation of gene transcription (Endocrinology 138: 863-870, 1997; Mol Endocrinol 11: 77-86, 1997; Endocrinology 123: 1281-1287, 1988; Breast Cancer 9: 296-302, 2002). Although the enzymes involved and the receptor have been well investigated, the transport mechanism of ligands such as estrone-3-sulfate across the plasma membrane remains to be clarified. Since estrone-3-sulfate is a hydrophilic compound, it would not be significantly transported across the plasma membrane by diffusion, though estrone and estradiol are diffusible (J Clin Endocrinol Metab 59: 1128-1132, 1984). Estrone-3-sulfate was detected in intact form in the cytosol fraction of MCF-7 cells after incubation with estrone-3-sulfate (Endocrinology 106: 1079-1086, 1980), and exhibited estrogenic effects (Ann N Y Acad Sci 464: 126-137, 1986; J Clin Endocrinol Metab 81: 1460-1464, 1996; J Steroid Biochem Mol Biol 73: 225-235, 2000). Accordingly, a specific transporter may be involved in the supply of estrone-3-sulfate to breast cancer cells which show hormone-dependent proliferation.

Several members of the organic anion transporter (OAT, SLC22A) (Pflugers Arch 440: 337-350, 2000) and organic anion transporting peptide (OATP, SLC21A, SLCO) (Biochim Biophys Acta 1609: 1-18, 2003, Pflugers Arch 447: 653-665, 2003) families are already known to transport estrone-3-sulfate. They are expressed mainly in liver, kidney, brain, intestine, and elsewhere, but their expression in breast cancer cells has not been reported. Their Km values for estrone-3-sulfate are in the range from 0.05 μM to 59 μM. Since plasma levels of estrone-3-sulfate fluctuate in the range of 1 to 10 nM in females (Hormone Res 27: 61-68, 1987), these or other transporters may play a role in the uptake of estrone-3-sulfate into cancer cells as the first step of hormone-dependent proliferation.

Recently, Pizzagalli et al. suggested the expression of a steroid sulfate transporter, OATP-B, in the human mammary gland (J Clin Endocrinol Metab 88: 3902-3912, 2003). However, OATP-B mRNA was not detected in estrogen-dependent MCF-7 and T-47D cells, while OATP-D and OATP-E mRNAs were found in these cells (J Clin Endocrinol Metab 88: 3902-3912, 2003). Since OATP-D and OATP-E exhibited transport activity for estrone-3-sulfate when their genes were transiently expressed in HEK293 cells (Tamai et al., Biochem. Biophys. Res. Commun., 273: 251-260, 2000), they may be involved in the transport of estrone-3-sulfate in cancer cells. However, the transporter-mediated uptake of estrone-3-sulfate in estrogen-dependent cells has not previously been examined. To fully understand the mechanism of proliferation of breast cancer cells, the mechanism of supply of estrogen to the cells must be clarified.

As to the proliferative inhibition of breast cancer cells which blocks upstream the signaling caused by binding of estrogen and its receptor in breast cancer cells, the following methods (blocks 1 to 4) are known, as shown in FIG. 1.

Block 1 (Inhibition of Estrogen Synthesis from Androgen)

Fadrozole: by selectively inhibiting aromatase which acts as a rate-limiting enzyme of estrogen synthesis to prevent conversion from androgen to estrogen after menopause, fadrozole decreases estrogen concentration in a living body and suppresses the proliferation of breast cancer.

Block 2 (Competitive Inhibition with Estrogen Receptors)

Tamoxifen, toremifene: by inhibiting estrogen receptors in breast cancer tissues, etc., competitively with estrogen and showing anti-estrogen effect, tamoxifen and toremifene exert anti-breast cancer effect.

Block 3 (Inhibition of Estrogen Synthesis from Estrone Sulfate)

Conjugated estrogens become active form in cells by estrogen sulfatase, and promote the proliferation of cancer cells. This deconjugating enzyme is inhibited.

Block 4 (Cytotoxic Effect Using Antibody to Membrane Protein)

Herceptin: some breast cancer cells highly express HER2 protein. With the use of the binding between this HER2 and its antibody, herceptin exerts anti-tumor effect caused by antibody-dependent cytotoxic effect.

As the above-mentioned methods, blocks 1 to 4, for inhibiting the proliferation of breast cancer cells, do not exert anti-tumor effect unless aromatase inhibitors, estrogen sulfatase inhibitors, or estrogen competitive inhibitors such as tamoxifen and toremifene are taken into breast cancer cells, they may have a problem in view of drug delivery. Further, when such inhibitors are taken into the cells, the methods may have a problem in view of adverse effect. The object of the present invention is to provide a method for screening a remedy for breast cancer which does not require uptake into cells and which shows excellent drug delivery property and extremely rare adverse effect.

The present inventors have conducted a keen study in order to attain the above-mentioned object, and examined the following block 5 (see FIG. 1) as a method for inhibiting the proliferation of breast cancer cells.

Block 5 (Inhibition of Transporter Activity)

By inhibiting the uptake of estrone-3-sulfate into cells, a source of estrogen, into estrogen-sensitive breast cancer cells, anti-tumor effect emerges.

The present inventors have found that when cultured human breast cancer cell line MCF-7 is cultured in the presence of estrone-3-sulfate and bromosulfophtalein (BSP), as a test substance, the uptake of estrone-3-sulfate into cells via estrone-3-sulfate transporters expressed on the cell surface is inhibited and the proliferation of MCF-7 cell line is suppressed, and thus the present invention has been completed.

### DISCLOSURE OF THE INVENTION

The present invention relates to: a method for screening a remedy for breast cancer comprising steps of: culturing a cell which expresses an estrone-3-sulfate transporter on its surface; measuring uptake of estrone-3-sulfate into the cultured cell in the presence of a test substance; and measuring/evaluating a level of inhibition of uptake activity of estrone-3-sulfate into the cell by the test substance (“1”); the method for screening a remedy for breast cancer according to “1”, wherein a level of inhibition of transporter activity is measured/evaluated by measuring/evaluating a concentration of estrone-3-sulfate in a cell (“2”); the method for screening a remedy for breast cancer according to “1”, wherein a level of inhibition of transporter activity is measured/evaluated by measuring/evaluating a level of cell proliferation (“3”); a method for screening a remedy for breast cancer comprising steps of: bringing estrone-3-sulfate and a test substance into contact with a cell membrane fraction isolated from a cell which expresses an estrone-3-sulfate transporter on its surface; and measuring/evaluating a level of inhibition of specific binding of estrone-3-sulfate to the cell membrane fraction by the test substance (“4”); a method for screening a remedy for breast cancer comprising steps of: isolating a cell membrane fraction from a cell which expresses an estrone-3-sulfate transporter on its surface; constructing an isolated cell membrane vesicle by making a vesicle from the cell membrane fraction; bringing estrone-3-sulfate and a test substance into contact with the isolated cell membrane vesicle; and measuring/evaluating uptake or a level of uptake inhibition of estrone-3-sulfate into the vesicle by the test substance (“5”); the method for screening a remedy for breast cancer according to any one of “1” to “5”, wherein the cell which expresses an estrone-3-sulfate transporter on its surface is a cultured human breast cancer cell line (“6”); the method for screening a remedy for breast cancer according to “6”, wherein the cultured human breast cancer cell line is an MCF-7 cell line, a T-47D cell line or a cell line derived therefrom (“7”); the method for screening a remedy for breast cancer according to any one of “1” to “7”, wherein a bulky anion compound which is an organic anion transporter inhibitor is used as a test substance (“8”); the method for screening a remedy for breast cancer according to “8”, wherein the bulky anion compound which is an organic anion transporter inhibitor is bromosulfophtalein, dehydroepiandrosterone sulfate, benzbromarone, DIDS, probenecid, sulfinpyrazone, bilirubin, statin HMGCoA reductase inhibitor, quinidine, quinine, digoxin, bile acids, thyroid hormone (T3, T4), or a synthetic oligopeptide (“9”); the method for screening a remedy for breast cancer according to any one of “1” to “7”, wherein a neutralizing antibody to an estrone-3-sulfate transporter is used as a test substance (“10”); the method for screening a remedy for breast cancer according to “10”, wherein the neutralizing antibody to an estrone-3-sulfate transporter is a specific antibody to an SLC transporter, OAT1, OAT2, OAT3, OAT4, OATP-A, OATP-B, OATP-C, OATP-D, OATP-E, OATP-F, OATP-8, NTCP, MRPs, or BCRP (“11”); the method for screening a remedy for breast cancer according to anyone of “1” to “11”, wherein the estrone-3-sulfate transporter is selected from an SLC transporter, OAT1, OAT2, OAT3, OAT4, OATP-A, OATP-B, OATP-C, OATP-D, OATP-E, OATP-F, OATP-8, NTCP, MRPs, and BCRP (“12”); and a remedy for breast cancer obtained by the method for screening according to any one of “1” to “12” (“13”).

### BRIEF DESCRIPTION OF DRAWINGS

FIG. 1 is a photograph of a frame format showing the molecular target of breast cancer.

FIG. 2 is a photograph showing the uptake inhibition of estrone-3-sulfate into breast cancer cells by BSP.

FIG. 3 is a set of views showing time course (a) and concentration dependence (b) of stimulation of proliferation of T-47D breast cancer cells by estrogen.

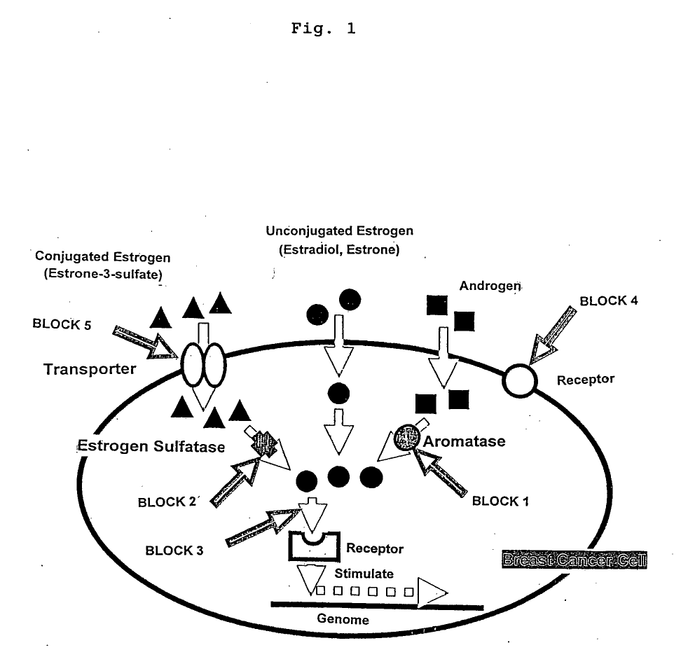

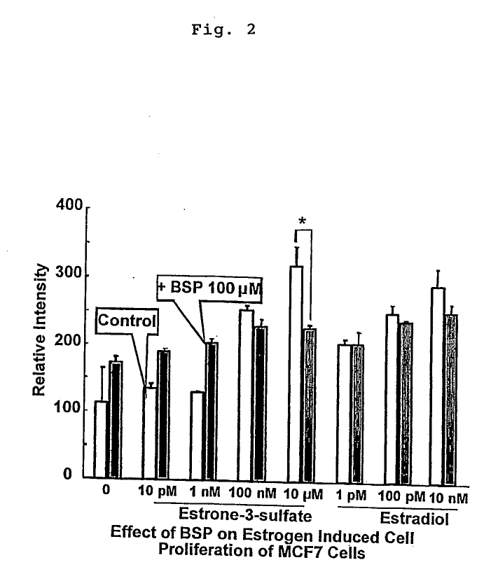

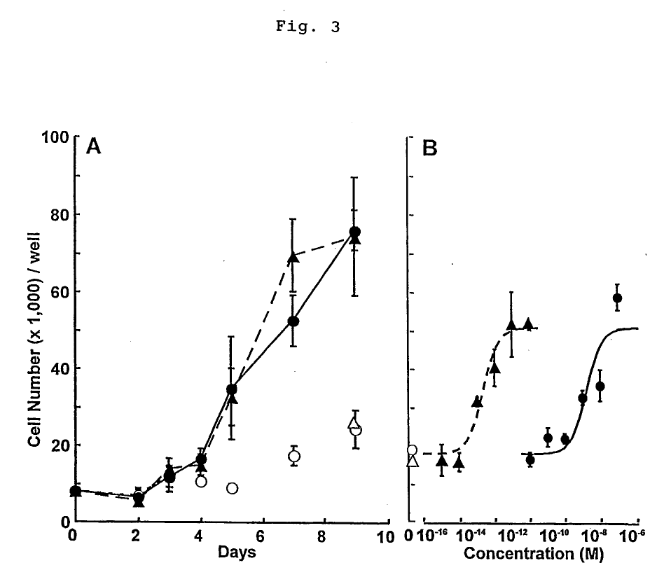

(a) T-47D cells were seeded at a density of 8,000 cells/well and cultured. 24 hours after seeding (day 1), estradiol (1 pM, closed triangles), estrone-3-sulfate (100 nM, closed circles), DMSO (0.1, open triangles) or water (open circles) was added. After the designated number of days, T-47D cells were trypsinized and counted.

(b) T-47D cells were proliferated in the presence of various concentrations of estradiol (closed triangles) or estrone-3-sulfate (closed circles), ranging from 10⁻¹⁶ to 10⁻¹¹ M, or from 10⁻¹¹ to 10⁻⁶ M, respectively. The cell numbers were counted at day 6 after seeding. Each value represents the mean±S.E.M. (n=3).

FIG. 4 is a view showing time course of [³H] estrone-3-sulfate uptake by T-47D cells.

Cultured T-47D cells were incubated at 37° C. over 35 min in medium containing [³H] estrone-3-sulfate (7.1 nM) with (open circles) or without (closed circles) 1 mM unlabeled estrone-3-sulfate. Each value represents the mean±S.E.M. (n=4). When the error bars are not shown, they are smaller than the symbols.

FIG. 5 is a view showing the effect of extracellular cations on [³H] estrone-3-sulfate uptake by T-47D cells.

The uptake rate of [³H] estrone-3-sulfate (7.1 nM) for 10 min was measured in the presence or absence of extracellular Na⁺. Extracellular Na⁺ was replaced with K⁺, Li⁺, or N-methylglucamine⁺ (NMG⁺). Each value represents the mean±S.E.M. (n=4).

FIG. 6 is a view showing the concentration dependence of uptake of estrone-3-sulfate by T-47D cells.

The uptake of estrone-3-sulfate at various concentrations, ranging from 5.1 nM to 50 μM, was measured at 37° C. for 10 min. The saturable uptake was obtained after subtraction of the uptake in the presence of excess of unlabeled estrone-3-sulfate (1 mM) and used for the evaluation of kinetic constants by nonlinear least-squares analysis. The inset shows an Eadie-Hofstee plot of the saturable uptake of estrone-3-sulfate. Each value represents the mean±S.E.M. (n=4). When the error bars are not shown, they are smaller than the symbols.

FIG. 7 is a view showing the concentration dependence of uptake of DHEAS by T-47D cells.

The uptake of DHEAS at various concentrations, ranging from 10.3 nM to 100 μM, was measured at 37° C. for 10 min. The saturable uptake was obtained after subtraction of the uptake in the presence of excess of unlabeled DHEAS (5 mM) and used for the evaluation of kinetic constants by nonlinear least-squares analysis. The inset shows an Eadie-Hofstee plot of the saturable uptake of DHEAS. Each value represents the mean±S.E.M. (n=4). When the error bars are not shown, they are smaller than the symbols.

FIG. 8 is a set of photographs showing the expression of OATP and OAT transporters in T-47D cells.

RT-PCR analysis was performed using mRNA obtained from T-47D cells. The reaction was performed as described in Materials and Methods. The arrows show the specific bands of OATP-D and OATP-E.

### BEST MODE OF CARRYING OUT THE INVENTION

The method for screening a remedy for breast cancer of the present invention is not particularly limited as long as it is a method comprising the steps of; culturing a cell which expresses an estrone-3-sulfate transporter on its surface, measuring uptake of estrone-3-sulfate into the cultured cell in the presence of a test substance, and measuring/evaluating a level of inhibition of transporter activity of estrone-3-sulfate into the cell by the test substance: a method comprising the steps of; bringing estrone-3-sulfate and a test substance into contact with a cell membrane fraction isolated from a cell which expresses an estrone-3-sulfate transporter on its surface, and measuring/evaluating a level of inhibition of specific binding of estrone-3-sulfate to the cell membrane fraction by the test substance: or a method comprising the steps of; isolating a cell membrane fraction from a cell which expresses an estrone-3-sulfate transporter on its surface; constructing an isolated cell membrane vesicle by making a vesicle from the cell membrane fraction; bringing estrone-3-sulfate and a test substance into contact with the isolated cell membrane vesicle; and measuring/evaluating uptake, or a level of uptake inhibition, of estrone-3-sulfate into the vesicle by the test substance. It is preferable to culture the cells in the presence of estrone-3-sulfate and a test substance with the use of a proliferation medium for the cell used, until at least cell proliferation is significantly observed (in the absence of a test substance) under the normal cell culture condition. Estrone-3-sulfate and a test substance can be brought into contact with a cell membrane fraction or with an isolated cell membrane vesicle by incubation in a proliferation medium for the cell used, or in an appropriate buffer solution.

As for the method for measuring/evaluating the level of inhibition of intracellular transporter activity of estrone-3-sulfate by the test substance described above, a method for measuring/evaluating the estrone-3-sulfate concentration in the cells, and a method for measuring/evaluating the level of cell proliferation are exemplified. In case where cultured human breast cancer cell lines such as MCF-7 cell line hereinafter described, KPL-1, MKL-F or cell lines derived therefrom are used, a method for measuring/evaluating the concentration of the test substance in the cells, and a method for measuring/evaluating the level of cell proliferation can be utilized, while in case where transformed cells expressing estrone-3-sulfate transporters are used, a method for measuring/evaluating the concentration of the test substance in the cells can be utilized. Further, it is possible to conduct measurement/evaluation by expressing estrone-3-sulfate transporter genes in Xenopus oocytes, etc., and by the electrophysiological method wherein changes in membrane potential associated with substrate transport via transporters are detected (electrical current is measured) with the use of two microelectrodes.

As a method for isolating cell membrane fractions from cells which express estrone-3-sulfate transporters on their surface, conventionally known methods such as a method of F. Pietri-Rouxel et al. (Eur. J. Biochem., 247, 1174-1179, 1997) can be used. Further, as a method for constructing isolated cell membrane vesicles by making vesicles from cell membrane fractions, conventionally known methods such as a method of J. E. Lever et al. (J. Biol. Chem., 252, 1990-1997, (1997)) can be used. Still further, as for a method for measuring a level of inhibition of specific binding of estrone-3-sulfate to the cell membrane fraction by the test substance, a method for measuring equilibrium dissociation constant KD from the steady state binding inhibition using radiolabeled estrone-3-sulfate is exemplified. As for a method for measuring uptake or a level of uptake inhibition of estrone-3-sulfate into the isolated cell membrane vesicle by the test substance, conventionally known methods such as a method of I. Tamai et al. (Biochim. Biophys. Acta, 1512, 273-284 (2001)) can be used as a method for measuring intracellular estrone-3-sulfate.

SLC transporters, OAT1, OAT2, OAT3, OAT4, OATP-A, OATP-B, OATP-C, OATP-D, OATP-E, OATP-F, OATP-8, NTCP, MRPs, BCRP are exemplified as preferable candidate transporters for the above-mentioned estrone-3-sulfate transporters.

As the cells which express estrone-3-sulfate transporters on their surface, cultured human breast cancer cell lines such as MCF-7 cell lines, T-47D cell lines, KPL-1, MKL-F or cell lines derived therefrom can be preferably used. Other than these cultured human breast cancer cell lines, transformed cells which express estrone-3-sulfate transporters can be used. There is no particular limitation for the origin of genes which encode the aforementioned SLC transporters, OAT1, OAT2, OAT3, OAT4, OATP-A, OATP-B, OATP-C, OATP-D, OATP-E, OATP-F, OATP-8, NTCP, MRPs, BCRP, which are used for making the transformed cells. Examples of such origin include human, canine, bovine, equine, goat, sheep, monkey, porcine, rabbit, rat, mouse, etc., however, genes derived from human are preferable.

Methods for expressing the above-mentioned genes or cDNAs encoding transporters in cells are not particularly limited, and it is possible to express them by introducing transporter genes into host cells by gene introduction methods described in a number of standard laboratory manuals, such as, by Davis et al. (BASIC METHODS IN MOLECULAR BIOLOGY, 1986), and by Sambrook et al. (MOLECULAR CLONING: A LABORATORY MANUAL, 2nd Ed., Cold Spring Harbor Laboratory Press, Cold Spring Harbor, N.Y., 1989), etc. Examples of those methods include calcium phosphate transfection, DEAE-dextran-mediated transfection, transfection, microinjection, cationic liposome-mediated transfection, electroporation, transduction, scrape loading, ballistic introduction and infection. It is preferable to introduce expression vectors containing transporter genes into the host cells, and it is more preferable to express transporters in the cells by infecting the expression vectors containing transporter genes with the host cells.

Specific examples of the expression vectors include: viral vectors such as adenoviral vectors used for transient expression in all cells (other than hemocytes) including nondividing cells (Science, 252, 431-434, 1991); retroviral vectors used for long-term expression in dividing cells (Microbiology and Immunology, 158, 1-23, 1992); adeno-associated virus vectors that can be introduced into nonpathogenic and nondividing cells, and is used for long-term expression (Curr. Top. Microbiol. Immunol., 158, 97-129, 1992); papovavirus vectors like SV40, vaccinia virus vectors; and liposome, but not limited thereto. Among them, adenoviral vectors, which can express genes in cells highly efficiently, are particularly preferable. In addition, transporter genes can be introduced into these expression vectors by ordinary methods, for example, expression vectors can be constructed by inserting transporter genes, etc., into downstream of appropriate promoters in these expression vectors. Further, the expression vectors may contain regulatory sequences such as enhancers or terminators which regulate expression other than transporter genes, marker genes, and IRES (internal ribosome entry site within mRNA) for standardizing expression amount of transporter per cell.

Examples of the host cells expressing transporters include insect cells such as drosophila S2, spodptera Sf9, and Vero cells, HeLa cells, CHO cells, WI-38 cells, BHK cells, COS-7 cells, MDCK cells, C127 cells, HKG cells, human kidney cell lines, CV-1 cells, LLC-MK2 cells, MDBK cells, MRC-5 cells, CaCo-2 cells, HT29 cells, human lymphoblast cells, oocytes of Xenopus, etc., and dhfr-deficient strains, HGPRT-deficient strains, ouabain-resistant strains thereof. Specifically, CHO-K1 (Chinese hamster ovary cells: ATCC CCL61), BHK (baby hamster kidney cells: ATCC CCL10), COS-7 (CV-1 Origin, SV-40 cells: ATCC CRL1651), Vero cells (African green monkey kidney cells: ATCC CCL81), human lymphoblast cells (IM-9, ATCC CCL159) are exemplified.

As for the test substance mentioned above, bulky anionic compounds which are organic anion transporter inhibitors are preferably exemplified as candidate compounds for remedies for breast cancer. More specific examples include: bromosulfophtalein, dehydroepiandrosterone sulfate (DHEAS), dehydroepiandrosterone, estradiol, taurocholic acid, benzbromarone, DIDS, probenecid, sulfinpyrazone, bilirubin, statin HMGCoA (3-hydroxy-3-methylglutaryl coenzyme A) reductase inhibitor (for example, pravastatin sodium, simvastatin, fluvastatin, etc.), quinidine, quinine, digoxin, bile acid (for example, taurocholate, cholate, etc.), thyroid hormone (T3, T4, etc.), and synthetic oligopeptides.

In addition, as transporters are membrane proteins expressed on the cell membrane surface, neutralizing antibodies to estrone-3-sulfate transporters may inhibit the target selectively from outside of the cells. Therefore, as for the test substance mentioned above, neutralizing antibodies to estrone-3-sulfate transporters are preferably exemplified as candidate substances for remedies for breast cancer. More specifically, antibodies which specifically recognize SLC transporters, OAT1, OAT2, OAT3, OAT4, OATP-A, OATP-B, OATP-C, OATP-D, OATP-E, OATP-F, OATP-8, NTCP, MRPs, BCRP, are exemplified. Examples of the specifically recognizing antibodies include monoclonal antibody, polyclonal antibody, single-stranded antibody, humanized antibody, chimeric antibody, bifunctional antibody which can recognize two epitopes simultaneously. These antibodies are produced by administrating cells which express estrone-3-sulfate transporters on their membrane surface or cell membrane fractions thereof to (preferably non-human) animals with commonly used protocols. For instance, in order to prepare monoclonal antibodies, any methods that bring antibodies produced by cultures of continuous cell lines, such as hybridoma (Nature 256, 495-497, 1975), trioma, human B cell hybridoma (Immunology Today 4, 72, 1983) and EBV-hybridoma (MONOCLONAL ANTIBODIES AND CANCER THERAPY, pp. 77-96, Alan R. Liss, Inc., 1985) can be used.

In addition, it is possible to use the preparation method of single stranded antibodies (U.S. Pat. Nos. 4,946,778; 5,260,203; 5,091,513; 5,455,030), the preparation method of humanized antibodies (U.S. Pat. No. 5,585,089; Nature, 321, 522-525, 1986; Protein Engineering, 4, 773-783, 1991), and the preparation method of chimeric antibodies (U.S. Pat. No. 4,816,567; Science, 229, 1202-1207, 1985; BioTechniques, 4, 214, 1986; Nature, 312, 643-646, 1984; Nature, 314, 268-270, 1985) to produce single stranded antibodies, humanized antibodies, and chimeric antibodies, respectively. Bifunctional antibodies can be produced by hybrid of two monoclonal cell lines which produce two related antibodies, or by chemical bonding of fragments of two antibodies. For example, there is a bifunctional antibody capable of binding to an estrone-3-sulfate transporter and a test peptide simultaneously.

The present invention is described below more specifically with reference to Examples, however, the technical scope of the present invention is not limited to these exemplification.

### EXAMPLE 1

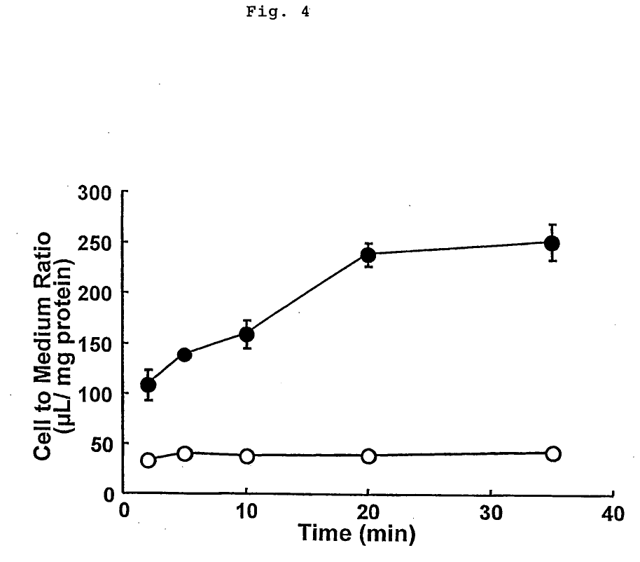

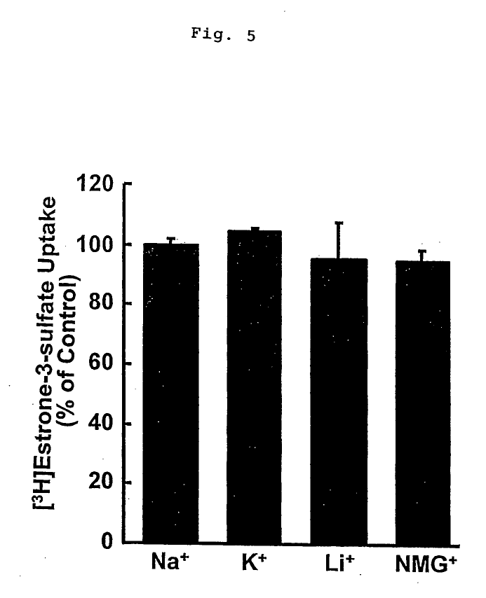

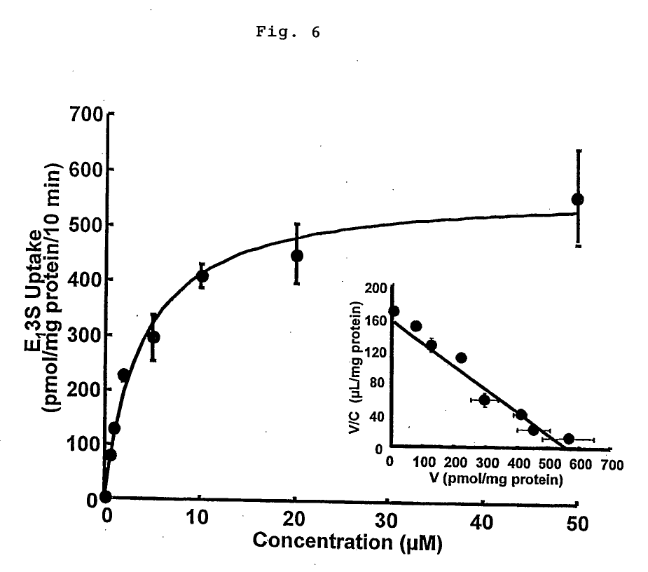

It was examined whether cell proliferation promoting effect on MCF-7 cells (ATCC-HTB22) by estrone-3-sulfate is inhibited by suppressing intracellular accumulation of estrone-3-sulfate. MCF-7 cells were seeded on a 96-well plate at a concentration of 8×10⁴ cells/cm², and cultured for 1 day. Then, a medium containing estrone-3-sulfate (Sigma Chemicals) at various concentrations, ranging from 10 pM to 10 μM, and 100 μM of bromosulfophtalein (BSP: Sigma Chemicals), and a medium containing estradiol (Sigma Chemicals) at various concentrations, ranging from 1 pM to 10 nM, and 100 μM of BSP were added respectively, and culture was conducted for 3 more days. Subsequently, cell amount was measured. The results of cell proliferation promoting effect by estrone-3-sulfate and estradiol are shown in FIG. 2.

As is clear from FIG. 2, in the presence of estrone-3-sulfate only and in the presence of estradiol only, which are positive controls, cell proliferation promoting effect was observed (open column). On the other hand, when cells were cultured in the presence of simultaneously added estrone-3-sulfate and 100 μM of BSP, or in the presence of simultaneously added estradiol and 100 μM of BSP, cell proliferation promoting effect was observed under the condition of low estrone-3-sulfate concentration. However, this is considered to be estrogen activity by BSP itself or metabolite thereof. This effect decreased relatively with the increase of estrogen concentration. Interestingly, cell proliferation promoting effect by BSP was inhibited when 10 μM of estrone-3-sulfate was added, while there was no change in the effect when estradiol was added. Accordingly, it is considered that the uptake of estrone-3-sulfate into the cells was inhibited by BSP, and that of estradiol, which is highly lipid-soluble, was not inhibited, and therefore there was no change in cell proliferation promoting effect. The above-mentioned fact suggests the importance of uptake transporters of estrone-3-sulfate in cell proliferation, and indicates the possibility of suppression of cell proliferation by inhibiting the transporters.

### EXAMPLE 2

1. Materials and Methods

(Materials)

[³H] Estrone-3-sulfate, ammonium salt (1702.0 GBq/mmol) and [³H] dehydroepiandrosterone sulfate (DHEAS, 2926.7 GBq/mmol) were purchased from PerkinElmer Life Science Products, Inc. T-47D cells were kindly provided by Professor Takuma Sasaki, Kanazawa University Cancer Research Institute. Fetal calf serum (FCS) was obtained from Invitrogen Corp. All other reagents were purchased from Sigma Chemicals and Wako Pure Chemical Industries.

(T-47D Cell Proliferation Assay)

T-47D cells were routinely proliferated in RPMI 1640 medium (Sigma Chemicals) containing phenol red and 10% FCS in a humidified incubator at 37° C. and 5% CO₂. For the proliferation assay, FCS was incubated with 0.5% dextran-coated charcoal (DCC) at 4° C. overnight, then the medium was decanted and DCC was filtered off (0.2 μm) to remove steroid hormones. This procedure was repeated three times. Then T-47D cells were seeded into 96-well plates at a density of 8,000 cells/well in phenol red-free RPMI 1640 medium containing 2.5% DCC-treated FCS. At 1 day after seeding, estradiol or estrone-3-sulfate was added at graded concentrations from stock solutions in water or dimethylsulfoxide (DMSO, 0.1%). The negative control was solvent only. At designated days after seeding, the cells were treated with trypsin, and cell numbers were counted.

(Inhibition of Uptake Activity of Estrone-3-Sulfate)

Transport experiments were performed as described previously (Tamai et al., 2001). After cultivation of T-47D cells in 15 cm dishes, the cells were harvested and suspended in the transport medium containing 125 mM NaCl, 4.8 mM KCl, 5.6 mM D-glucose, 1.2 mM CaCl₂, 1.2 mM KH₂PO₄, 1.2 mM MgSO₄ and 25 mM Hepes, adjusted to pH 7.4. The cell suspension and a solution containing a radiolabeled test compound in the transport medium were separately incubated at 37° C. for 20 min, then transport was initiated by mixing them. At appropriate times, 150 μL aliquots of the mixture were withdrawn and the cells were separated from the transport medium by centrifugal filtration through a layer of a mixture solution of silicone oil (SH550, Toray Dow Corning Co.) and liquid paraffin (Wako Pure Chemical Industries) with a density of 1.03. Each cell pellet was solubilized in 3 N KOH, and neutralized with HCl. Then, the associated radioactivity was measured by means of a liquid scintillation counter using Clearsol-1 (Nacalai tesque) as a liquid scintillation fluid.

(Reverse Transcription PCR Method)

Expression of OATP and OAT transporters in T-47D cells was examined by RT-PCR method. Single-stranded cDNA was constructed using an oligo (dT) primer (Invitrogen Corp.). The following specific primers were used.

| | |
|---|---|
| OATP-A: | |
| (F) 5′-AAACAAGCTGCCCACATAGG-3′ | (SEQ ID NO: 1) |
| (R) 3′-CAGCAAGACAAGCTGACAGA-5′ | (SEQ ID NO: 2) |
| OATP-B: | |
| (F) 5′-CCTGCCGCTCTTCTTTATCGG-3′ | (SEQ ID NO: 3) |
| (R) 3′-ACCAGATGGCTGCACGTTG-5′ | (SEQ ID NO: 4) |
| OATP-C: | |
| (F) 5′-CACTTGGAGGCACCTCACA-3′ | (SEQ ID NO: 5) |
| (R) 3′-ACAAGCCCAAGTAGACCCTT-5′ | (SEQ ID NO: 6) |
| OATP-D: | |
| (F) 5′-CAGGCCATGCTCTCCGAAA-3′ | (SEQ ID NO: 7) |
| (R) 3′-AGCCACCACTGCAATCTCC-5′ | (SEQ ID NO: 8) |
| OATP-E: | |
| (F) 5′-CCCTGGGAATCCAGTGGATTG-3′ | (SEQ ID NO: 9) |
| (R) 3′-AGCAGGCTATGGCAAAGAAGAG-5′ | (SEQ ID NO: 10) |
| OATP-F: | |
| (F) 5′-GGAAATTCCTCAGGCATAGTGG-3′ | (SEQ ID NO: 11) |
| (R) 3′-CTGGGATTCCTGCAAGAACTC-5′ | (SEQ ID NO: 12) |
| OATP-8: | |
| (F) 5′-GGGAATCATAACCATTCCTACGG-3′ | (SEQ ID NO: 13) |
| (R) 3′-GAGGATTTGCATCCTGCTAGAC-5′ | (SEQ ID NO: 14) |
| OAT1: | |
| (F) 5′-CTGATGGCTTCTCACAACAC-3′ | (SEQ ID NO: 15) |
| (R) 3′-CCGACTCAATGAAGAACCAG-5′ | (SEQ ID NO: 16) |
| OAT2: | |
| (F) 5′-GCTGGTTTTACCATCATCGT-3′ | (SEQ ID NO: 17) |
| (R) 3′-GACTCAGGCCGTAATAGGAG-5′ | (SEQ ID NO: 18) |
| OAT3: | |
| (F) 5′-AAGTGACCTGTTCCGGATAC-3′ | (SEQ ID NO: 19) |
| (R) 3′-CCATACCTGTTTGCCTGATG-5′ | (SEQ ID NO: 20) |
| OAT4: | |
| (F) 5′-GGCGTTATCTCCATTGCTTC-3′ | (SEQ ID NO: 21) |
| (R) 3′-GAGATTGGAACCCAGTCTCT-5′ | (SEQ ID NO: 22) |

The reaction was performed 35 cycles of 94° C. for 30 sec, 58° C. (OATPs) or 56° C. (OATs) for 30 sec, 72° C. for 30 sec and final elongation at 72° C. for 10 min the presence of deoxynucleotides and tag polymerase (Takara Bio Inc.). PCR products were analyzed by means of 1% agarose gel (w/v) electrophoresis and the gel was stained with ethidium bromide to visualize bands.
(Analytical Methods)

Cellular protein content was determined according to the method of Bradford by using a BioRad protein assay kit (Hercules) with bovine serum albumin as the standard (Bradford, 1976). To estimate kinetic parameters for saturable transport, the uptake rate (v) was fitted to the following equation by means of nonlinear least-squares regression analysis using KaleidaGraph™ (Synergy).
v=V max*s/(Km+s)
wherein v and s are the uptake rate and concentration of substrate, respectively, and Km and Vmax represent the half-saturation concentration (Michaelis constant) and the maximum transport rate, respectively. All data were expressed as means±S.E.M., and statistical analysis was performed by the use of Student's t test with P<0.05 as the criterion of significance. Cell-to-medium ratio was obtained by dividing the cellular uptake amount by the concentration of test compound in the uptake medium.
2. Results (Effect of Estradiol and Estrone-3-Sulfate on the Proliferation of T-47D Cells)

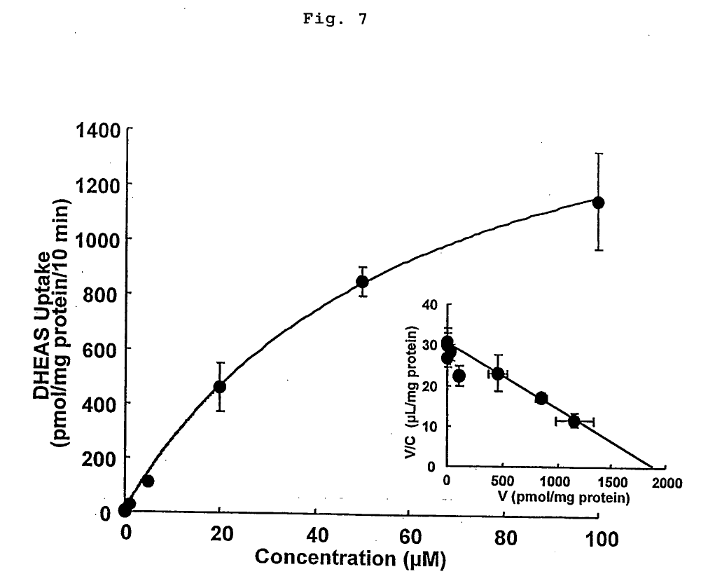

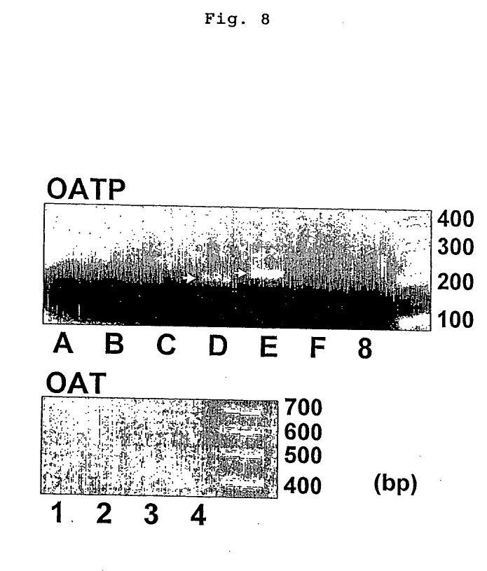

To elucidate the effect of estrone-3-sulfate on the proliferation of T-47D cells, the cells were treated with estradiol and estrone-3-sulfate. Inline with a previous study using estrogen-dependent MCF-7 cells (Billich et al., 2000), the proliferation of T-47D cells was stimulated both by 1 pM estradiol and by 100 nM estrone-3-sulfate compared with the cells treated with solvent alone (FIG. 3A). When evaluated at 6 days after seeding, the estrogen-dependent stimulation was increased in a concentration-dependent manner (FIG. 3B). The values of median effective concentration (EC₅₀) of estradiol and estrone-3-sulfate were evaluated as approximately 22.5 fM and 17.1 nM, respectively, by nonlinear regression analysis.

(Uptake of Estrone-3-Sulfate by T-47D Cells)

FIG. 4 shows the time course of the uptake of [³H] estrone-3-sulfate (7.1 nM) by T-47D cells in the presence or absence of unlabeled 1 mM estrone-3-sulfate. The uptake of [³H] estrone-3-sulfate increased linearly over 20 min. In the presence of unlabeled 1 mM estrone-3-sulfate, no significant increase in the uptake of [³H] estrone-3-sulfate were observed over 35 min (FIG. 4), suggesting that estrone-3-sulfate was hardly taken up into T-47D cells by simple diffusion. Accordingly, in the following experiments, uptake of estrone-3-sulfate by T-47D cells was kinetically analyzed over 10 min. Furthermore, the apparent uptakes were analyzed after subtraction of the uptake in the presence of unlabeled estrone-3-sulfate (non-saturable uptake), to take account of adsorption on the cell membrane and uptake by simple diffusion.

To characterize the transport mechanism of estrone-3-sulfate, replacement of Na⁺ with various cations was examined (FIG. 5). When Na⁺ was replaced with K⁺, Li⁺, or N-methylglucamine⁺, the uptake of [³H] estrone-3-sulfate was comparable to that in the presence of Na⁺, suggesting a Na⁺-independent transport mechanism.

To obtain the kinetic parameters of the uptake of estrone-3-sulfate by T-47D cells, the concentration dependence of estrone-3-sulfate uptake was examined. The uptake of estrone-3-sulfate after subtraction of the value in the presence of 1 mM estrone-3-sulfate was saturable, as shown in FIG. 6. In Eadie-Hofstee plot analysis, a single straight line was obtained, suggesting that a single saturable mechanism is involved kinetically. Nonlinear regression analysis yielded Km and Vmax values (mean±S.E.M.) of 3.9±0.78 μM and 576.3±32.9 pmol/mg protein/10 min, respectively.

(Inhibition of Uptake Activity of Estrone-3-Sulfate)

To characterize the substrate specificity of the transport system, the inhibitory effects of hormones, their conjugated metabolites, and various organic compounds on [³H] estrone-3-sulfate uptake by T-47D cells were examined. The results are shown in (Table 1). In Table 1, values in control (%) are indicated as the ratio in comparison to uptake amount of estrone-3-sulfate in control which was set at 100%. Each value represents the mean±S.E.M. (n=4), and *P<0.05 indicates comparison to control (Student's test).

TABLE 1

| Inhibitors | Concentration (μM) | Control (%) |
|---|---|---|
| Control | | 100.0 ± 4.7 |
| Estrone-3-sulfate | 5 | 34.7 ± 4.3 * |
| | 50 | 6.6 ± 0.9 * |
| Dehydroepiandrosterone sulfate | 5 | 66.4 ± 5.7 * |
| | 50 | 27.0 ± 7.6 * |
| Estradiol-17β-glucuronide | 5 | 73.8 ± 8.7 |
| | 50 | 90.4 ± 11.5 |
| Estrone | 5 | 78.8 ± 3.3 * |
| | 50 | 64.8 ± 7.5 * |
| Dehydroepiandrosterone | 5 | 75.6 ± 3.8 * |
| | 50 | 44.8 ± 8.0 * |
| Estradiol | 5 | 76.9 ± 4.8 * |
| | 50 | 45.7 ± 5.3 * |
| Bromosulfophtalein | 100 | 18.9 ± 2.8 * |
| | 1000 | 14.2 ± 1.2 * |
| Taurocholic acid | 100 | 69.3 ± 2.7 * |
| | 1000 | 24.1 ± 2.3 * |
| Probenecid | 100 | 103.2 ± 10.2 |
| | 1000 | 79.1 ± 3.8 * |
| Penicillin G | 100 | 83.2 ± 2.0 * |
| | 1000 | 75.9 ± 10.5 |
| Salicylate | 100 | 94.9 ± 6.0 |
| | 1000 | 82.7 ± 7.3 |
| p-Aminohippuric acid | 100 | 87.8 ± 1.5 |
| | 1000 | 112.7 ± 18.6 |
| Tetraethylammonium | 100 | 87.4 ± 1.5 * |
| | 1000 | 112.7 ± 18.6 |
| Control (DMSO 1%) | | 100 ± 9.7 |
| Cyclosporin A (DMSO 1%) | 10 | 112.2 ± 8.3 |
| | 100 | 90.3 ± 3.7 |
| Digoxin (DMSO 1%) | 100 | 90.3 ± 3.5 |
| | 1000 | 94.8 ± 4.4 |

As a result, unlabeled estrone-3-sulfate itself exhibited the strongest inhibitory effect (to 34.7% and 6.6% of the control at 5 μM and 50 μM, respectively). The inhibitory effect of DHEAS was significant (to 44.8% of the control at 50 μM), but weaker than that of estrone-3-sulfate. A glucuronide conjugate, estradiol-17β-glucuronide, had no effect at 50 μM. Unconjugated steroid hormones all significantly inhibited the uptake of [³H] estrone-3-sulfate. However, their affinities were lower than that of sulfate conjugates, estrone-3-sulfate and DHEAS. Among other organic compounds examined, bromosulfophthalein and taurocholic acid were inhibitory at 100 μM (to 18.9% and 69.3% of the control, respectively). Other organic compounds, salicylate, p-aminohippuric acid, tetraethylammonium, cyclosporin A, and digoxin had no effect, while probenecid and benzyl penicillin showed slight inhibitory effects.

In postmenopausal women, androgens and their sulfated conjugates play an important role in the progression of breast cancer as a source of active estrogen. Since DHEAS is present at high concentration (about 1 μM) in most postmenopausal patients, it is a major precursor (Worgul et al., 1982). Therefore, it was examined whether DHEAS is transported via a specific transport mechanism. The uptake of DHEAS increased linearly over 30 min, and significantly decreased in the presence of 5 mM unlabeled DHEAS (data not shown). The uptake of DHEAS, after subtraction of the uptake in the presence of 5 mM unlabeled DHEAS (nonspecific uptake), was saturable (FIG. 7). Eadie-Hofstee analysis of DHEAS uptake gave a single straight line, as seen in the case of estrone-3-sulfate (FIG. 7, inset), and the Km and Vmax values were calculated as 62.5±21.1 μM and 1893±315.7 pmol/mg protein/10 min, respectively.

To identify the transporters which mediate the uptake of estrone-3-sulfate and DHEAS in T-47D cells, the expression of OATP and OAT transporters, which could potentially transport estrone-3-sulfate, was determined by RT-PCR analysis. Among the OATPs and OATs tested, the expression of OATP-D and OATP-E was detected in T-47D cells by using specific primers (FIG. 8), while no band was detected by PCR without RT reaction (data not shown). Thus, OATP-D and OATP-E may be candidates for mediating the uptake of estrone-3-sulfate and DHEAS in estrogen-dependent breast cancer cells.

3. Discussion

Estrone-3-sulfate is a major circulating estrogen, and participates in the progression of estrogen-dependent breast cancer (Pasqualini J R, Gelly C, Nguyen B L and Vella C (1989) Importance of estrogen sulfates in breast cancer. J Steroid Biochem 34: 155-163). It is subject to desulfation by steroid sulfatase, followed by conversion to biologically active estrogen in tumor tissues. Since steroid sulfatase was detected in the cytoplasm of breast cancer cells (Saeki T, Takashima S, Sasaki H, Hanai N and Salomon D S (1999) Localization of estrone sulfatase in human breast carcinomas. Breast Cancer 6: 331-337), estrone-3-sulfate must be transported into the cells across the plasma membrane prior to desulfation. In view of the hydrophilicity of estrone-3-sulfate, the internalization of estrone-3-sulfate is likely to be mediated by a specific transport mechanism, whereas hydrophobic unconjugated steroid hormones seem simply to diffuse into the cells (Verheugen C, Pardridge W M, Judd H L and Chaudhuri G (1984) Differential permeability of uterine and liver vascular beds to estrogens and estrogen conjugates. J Clin Endocrinol Metab 59: 1128-1132) So, it was examined in the present study whether there is a specific transport system which mediates the uptake of estrone-3-sulfate and DHEAS into T-47D breast cancer cells, which are regarded as model cells that show estrogen-dependent proliferation.

First of all, the estrogen dependence of the cell proliferation was confirmed. Treatment with estrone-3-sulfate, or estradiol, increased T-47D cell proliferation with an EC₅₀ value of estrone-3-sulfate of 17.1 nM, which is close to the physiological plasma concentration of estrone-3-sulfate. Since estrone-3-sulfate itself induces a low direct biological response at the estrogen receptor (Kuiper et al., 1997), the results suggested the internalization of estrone-3-sulfate across the cell membrane followed by conversion to active unconjugated estrogen in the cells. Accordingly, this study was particularly focused on the uptake of the sulfate conjugate of estrogen by T-47D cells, as the first step in the elucidation of estrogen activity.

The time course of estrone-3-sulfate uptake by T-47D cells suggested that the uptake was mediated by a specific transporter(s), since the uptake in the presence of a high concentration of estrone-3-sulfate (1 mM) was significantly decreased (FIG. 4). The specific uptake of estrone-3-sulfate was not affected by replacement of Na⁺ with Li⁺, K⁺ or N-methylglucamine⁺ (FIG. 5). Members of the OATP and OAT families are candidate substances for Na⁺-independent estrone-3-sulfate uptake transporters. The Km value for estrone-3-sulfate uptake by T-47D cells was calculated to be 3.9±0.78 μM (FIG. 6), which is consistent with the known range of Km values of OATPs and OATs of 0.05 μM to 59 μM (Tamai I, Nozawa T, Koshida M, Nezu J, Sai Y and Tsuji A (2001) Functional characterization of human organic anion transporting polypeptide B (OATP-B) in comparison with liver-specific OATP-C. Pharm Res 18: 1262-1269; Kusuhara H, Sekine T, Utsunomiya-Tate N, Tsuda M, Kojima R, Cha S H, Sugiyama, Y, Kanai Y, and Endou H (1999) Molecular cloning and characterization of a new multispecific organic anion transporter from rat brain. J Biol Chem 274: 13675-13680; Cha S H, Sekine T, Kusuhara H, Yu E, Kim J Y, Kim D K, Sugiyama Y, Kanai Y and Endou H (2000) Molecular cloning and characterization of multispecific organic anion transporter 4 expressed in the placenta. J Biol Chem 275: 4507-4512).

To further characterize the transport mechanism and to identify the transporter involved, an inhibition study on the uptake of estrone-3-sulfate by T-47D cells was performed. The inhibitory effects of sulfate conjugates of steroid hormones such as estrone-3-sulfate and DHEAS were stronger than those of unconjugated steroid hormones, while a glucuronide conjugate was not inhibitory. Thus, a sulfate moiety might confer high affinity for the transporter, while a glucuronide moiety might be hardly recognized. Since sulfate conjugates have lower ionization constant (pKa) values compared with unconjugated steroids orglucuronide conjugates, these results suggested that an anionic moiety is essential for substrate recognition by the transporter. Because the plasma concentration of sulfate conjugates of steroid hormones is higher than that of unconjugated hormones or their glucuronide conjugates, the transporter should act effectively in the supply of estrogens to breast tumors. Moreover, the uptake of DHEAS was also saturable in the T-47D cells, and the Km value was calculated as 62.5±21.1 μM, which is much higher than that of estrone-3-sulfate (3.9 μM, FIGS. 6 and 7). Since the plasma concentrations of estrone-3-sulfate and DHEAS are approximately 1 to 10 nM and 1 μM, respectively (Honjo H, Kitawaki J, Itoh M, Yasuda J, Iwasaku K, Urabe M, Naitoh K, Yamamoto T, Okada H, Ohkubo T and Nambara T (1987) Hormone Res 27: 61-68; Worgul T J, Santen R J, Samojlik E and Wells S A (1982) How effective is surgical adrenalectomy in lowering steroid hormone concentrations? J Clin Endocrinol Metab 54: 22-26), breast cancer cells may effectively take them up without saturation.

Some members of OATPs, OAT3 and OAT4 can transport sulfate conjugates of steroid hormones (Hagenbuch B and Meier P J (2003a) Biochim Biophys Acta 1609: 1-18, Hagenbuch B and Meier P J (2003b) Pflugers Arch 447: 653-665; Saeki T, Takashima S, Sasaki H, Hanai N and Salomon D S (1999) Localization of estrone sulfatase in human breast carcinomas. Breast Cancer 6: 331-337; Kusuhara H, Sekine T, Utsunomiya-Tate N, Tsuda M, Kojima R, Cha S H, Sugiyama, Y, Kanai Y, and Endou H (1999) Molecular cloning and characterization of a new multispecific organic anion transporter from rat brain. J Biol Chem 274: 13675-13680). Among them, OATP-B and OAT4 recognize sulfate conjugate with Km values of 9.04 μM and 1.01 μM, respectively, while they have no affinity for glucuronide conjugates of steroid hormones (Tamai I, Nozawa T, Koshida M, Nezu J, Sai Y and Tsuji A (2001) Functional characterization of human organic anion transporting polypeptide B (OATP-B) in comparison with liver-specific OATP-C. Pharm Res 18: 1262-1269; Cha S H, Sekine T, Kusuhara H, Yu E, Kim J Y, Kim D K, Sugiyama Y, Kanai Y and Endou H (2000) Molecular cloning and characterization of multispecific organic anion transporter 4 expressed in the placenta. J Biol Chem 275: 4507-4512). Since the uptake of [³H] estrone-3-sulfate was not inhibited by glucuronide conjugates in T-47D cells, the functional characteristics of OATP-B or OAT4 are consistent with the uptake mechanism in T-47D cells as to the substrate recognition of steroid conjugates. On the other hand, since OATP-A, OATP-C, OATP-8, and OAT3 transport both sulfate and glucuronide conjugates, these transporters are not likely to be involved in the transport of estrone-3-sulfate in T-47D cells (Hagenbuch B and Meier P J (2003a) Biochim Biophys Acta 1609: 1-18, Hagenbuch B and Meier P J (2003b) Pflugers Arch 447: 653-665; Kusuhara H, Sekine T, Utsunomiya-Tate N, Tsuda M, Kojima R, Cha S H, Sugiyama, Y, Kanai Y, and Endou H (1999) Molecular cloning and characterization of a new multispecific organic anion transporter from rat brain. J Biol Chem 274: 13675-13680). To obtain insight into the nature of the key transporter, the expression of various human OATPs and OATs was examined by RT-PCR analysis (FIG. 8). The expression of only OATP-D and OATP-E was detected, in agreement with a previous report (Pizzagalli F, Varga Z, Huber R D, Folkers G, Meier P J and St-Pierre M V (2003) Identification of steroid sulfate transport processes in the human mammary gland. J Clin Endocrinol Metab 88: 3902-3912). Since OATP-D and OATP-E exhibited transport activity for estrone-3-sulfate when they were transiently expressed in HEK293 cells (Tamai I, Nezu J, Uchino H, Sai Y, Oku A, Shimane M and Tsuji A (2000) Molecular identification and characterization of novel members of the human organic anion transporter (OATP) family. Biochem Biophys Res Commun 273: 251-260), they may take part in the uptake of estrone-3-sulfate across the crll membrane in T-47D cells. However, since their functional characteristics have not been well clarified, the transporter involved in the uptake of estrone-3-sulfate by T-47D cells could not be identified in the present study. It has also been reported that BCRP (ABCG2) and OSTalpha-OSTbeta can accept estrone-3-sulfate as a substrate (Suzuki T, Moriya T, Ishida T, Kimura M, Ohuchi N and Sasano H (2002) In situ production of estrogens in human breast carcinoma. Breast Cancer 9: 296-302; Doyle L A, Yang W, Abruzzo L V, Krogmann T, Gao Y, Rishi A K and Ross D D (1998) A multidrug resistance transporter from human MCF-7 breast cancer cells. Proc Natl Acad Sci USA 95: 15665-15670; Tsuruoka S, Ishibashi K, Yamamoto H, Wakaumi M, Suzuki M, Schwartz G J, Imai M and Fujimura A (2002) Functional analysis of ABCA8, a new drug transporter. Biochem Biophys Res Commun 298: 41-45; Imai Y, Asada S, Tsukahara S, Ishikawa E, Tsuruo T and Sugimoto Y (2003) Breast cancer resistance protein exports sulfated estrogens but not free estrogens. Mol Pharmacol 64: 610-618; Seward D J, Koh A S, Boyer J L and Ballatori N (2003) Functional complementation between a novel mammalian polygenic transport complex and an evolutionarily ancient organic solute transporter, OSTalpha-OSTbeta. J Biol Chem 278: 27473-27482). Therefore, transporters other than OATPs and OATs might also be involved in the uptake of estrone-3-sulfate in T-47D cells.

The above-mentioned Examples clearly demonstrated that estrone-3-sulfate is taken up by estrogen-dependent T-47D cells via a specific transport mechanism. This is the first demonstration that the major circulating estrogens, estrone-3-sulfate and DHEAS, are supplied to estrogen-dependent breast cancer cells via a specific transporter. Although further study is required to identify the transporter in T-47D cells, such a transporter molecule would be a novel molecular target for therapy of estrogen-dependent breast cancer.

### INDUSTRIAL APPLICABILITY

The present invention makes it possible to screen a remedy for breast cancer which does not require uptake into cells unlike aromatase inhibitors, estrogen sulfatase inhibitors, or estrogen competitive inhibitors such as tamoxifen and toremifene, and which shows excellent drug delivery property and extremely rare adverse effect.

1. A method for screening a remedy for breast cancer comprising steps of: culturing a cell which expresses an estrone-3-sulfate transporter on its surface; measuring uptake of estrone-3-sulfate into the cultured cell in the presence of a test substance; and measuring/evaluating a level of inhibition of uptake activity of estrone-3-sulfate into the cell by the test substance.

2. The method for screening a remedy for breast cancer according to claim 1, wherein a level of inhibition of transporter activity is measured/evaluated by measuring/evaluating a concentration of estrone-3-sulfate in a cell.

3. The method for screening a remedy for breast cancer according to claim 1, wherein a level of inhibition of transporter activity is measured/evaluated by measuring/evaluating a level of cell proliferation.

4. A method for screening a remedy for breast cancer comprising steps of: bringing estrone-3-sulfate and a test substance into contact with a cell membrane fraction isolated from a cell which expresses an estrone-3-sulfate transporter on its surface; and measuring/evaluating a level of inhibition of specific binding of estrone-3-sulfate to the cell membrane fraction by the test substance.

5. A method for screening a remedy for breast cancer comprising steps of: isolating a cell membrane fraction from a cell which expresses an estrone-3-sulfate transporter on its surface; constructing an isolated cell membrane vesicle by making a vesicle from the cell membrane fraction; bringing estrone-3-sulfate and a test substance into contact with the isolated cell membrane vesicle; and measuring/evaluating uptake or a level of uptake inhibition of estrone-3-sulfate into the vesicle by the test substance.

6. The method for screening a remedy for breast cancer according to any one of claims 1 to 5, wherein the cell which expresses an estrone-3-sulfate transporter on its surface is a cultured human breast cancer cell line.

7. The method for screening a remedy for breast cancer according to claim 6, wherein the cultured human breast cancer cell line is an MCF-7 cell line, a T-47D cell line or a cell line derived therefrom.

8. The method for screening a remedy for breast cancer according to any one of claims 1 to 7, wherein a bulky anion compound which is an organic anion transporter inhibitor is used as a test substance.

9. The method for screening a remedy for breast cancer according to claim 8, wherein the bulky anion compound which is an organic anion transporter inhibitor is bromosulfophtalein, dehydroepiandrosterone sulfate, dehydroepiandrosterone, estradiol, taurocholic acid, benzbromarone, DIDS, probenecid, sulfinpyrazone, bilirubin, statin HMGCoA reductase inhibitor, quinidine, quinine, digoxin, bile acids, thyroid hormone (T3, T4), or a synthetic oligopeptide.

10. The method for screening a remedy for breast cancer according to any one of claims 1 to 7, wherein a neutralizing antibody to an estrone-3-sulfate transporter is used as a test substance.

11. The method for screening a remedy for breast cancer according to claim 10, wherein the neutralizing antibody to an estrone-3-sulfate transporter is a specific antibody to an SLC transporter, OAT1, OAT2, OAT3, OAT4, OATP-A, OATP-B, OATP-C, OATP-D, OATP-E, OATP-F, OATP-8, NTCP, MRPs, or BCRP.

12. The method for screening a remedy for breast cancer according to any one of claims 1 to 11, wherein the estrone-3-sulfate transporter is selected from an SLC transporter, OAT1, OAT2, OAT3, OAT4, OATP-A, OATP-B, OATP-C, OATP-D, OATP-E, OATP-F, OATP-8, NTCP, MRPs, and BCRP.

13. A remedy for breast cancer obtained by the method for screening according to any one of claims 1 to 12.
